(19) United States
(12) Patent Application Publication
(10) Pub. No.: US 2014/0251334 A1
(43) Pub. Date: Sep. 11, 2014

(54) SLEEP APNEA MASK COVER

(71) Applicant: Beth Kramer, Swanton, OH (US)

(72) Inventor: Beth Kramer, Swanton, OH (US)

(21) Appl. No.: 14/196,057

(22) Filed: Mar. 4, 2014

Related U.S. Application Data
(60) Provisional application No. 61/772,605, filed on Mar. 5, 2013.

(51) Int. Cl. A61M 16/06; A61M 16/08; A61M 16/00

(52) U.S. Cl. CPC A61M 16/0616; A61M 16/0057; A61M 16/0683; A61M 16/0816; A61M 16/0875
USPC 128/205.25

(57) ABSTRACT

The present invention is related to a cover for a CPAP mask formed from an insulating material. The cover is adapted to fit over a frame of a CPAP mask without contacting a wearer of the CPAP mask and to mitigate the formation of condensation on an interior of the CPAP mask.

### CROSS-REFERENCE TO RELATED APPLICATION

This application claims the benefit of priority of U.S. Provisional Patent Application Ser. No. 61/772,605 filed on Mar. 5, 2013 hereby incorporated herein by reference in its entirety.

### FIELD OF THE INVENTION

The present invention relates generally to the field of respiration, breathing, and/or ventilation masks. In particular, the invention relates to an insulating cover for masks used in the treatment of sleep apnea.

### BACKGROUND OF THE INVENTION

Obstructive sleep apnea syndrome (commonly referred to as obstructive sleep apnea, OSA, and/or sleep apnea) is a medical condition that includes a symptom of repeated and prolonged episodes of cessation of breathing throughout the night during sleep. This is generally a condition in which a person's airway becomes blocked or restricted due to the over-relaxation of the muscles and tissues in the throat. A restrictive air supply impairs the oxygen flow to the lungs and, in turn, to the brain.

An individual with OSA may stop breathing, or suffer an apnea, on a plurality of occasions of typically 10 to 30 times an hour during sleep. The apneas are generally followed by sudden reflexive attempts to breathe. As a result, the individual suffering from OSA experiences fragmented sleep. Such sleep may result in daytime drowsiness, headaches, weight gain, limited attention span, memory loss, lethargy, inability to maintain concentration and/or depression. OSA has also been linked to more serious medical conditions, such as increased hypertension and greater risk of stroke, heart disease, and diabetes, if left untreated.

Effective treatment requires the airway to remain open during sleep. Modern respiratory therapy utilizes a “continuous positive airway pressure” (CPAP) machine and a ventilation interface worn during sleep in order to keep the airways open. A variety of CPAP machines can provide this treatment. BiPAP and VPAP are two specialized examples of CPAP machines that vary the pressure received based on the patient's breathing patterns. All positive airway pressure machines use an interface to deliver ambient air, under pressure, to the patient's airway. This interface usually consists of a nasal mask, a full-face mask (covering both the nose and mouth), or nasal pillows held onto the patient's face with straps, or headgear, and connected to the CPAP machine by way of an air hose. The nasal mask and full-face mask employ a forehead pad that rests against the patient's forehead to stabilize the mask and to position a seal of the portion of the mask that covers the nose over the nose.

No matter what type of OSA interface is used, a problem wearers have is that condensation may form on the inside of the mask. Condensation may occur when the ambient temperature is relatively cool as compared to the temperature of the breath inside the mask (typically about 98.6° F.). If enough condensation is generated and not removed from the inside of the mask, the breathing of the wearer is interrupted. Furthermore, the condensation must be removed from the mask, thus further interrupting the sleep of the wearer.

CPAP covers produced and sold under the PAD A CHEEK® mark by Pad A Cheek LLC and the SLEEPWEAVER™ mark by Circadiance LLC are designed to cover a CPAP mask and to contact the mask wearer. Both masks are designed to prevent the seal of the CPAP mask from contacting the wearer for comfort and/or in the event of an allergy related to the material the seal is formed from. The SLEEPWEAVER™ cover is inflatable and forms a seal between itself and a face of the wearer to hold the CPAP mask in place. The SLEEPWEAVER™ cover is specifically not adapted to militate against condensation forming in the CPAP mask, rather it allows condensation to form but wicks moisture out of the mask.

It would be desirable to develop a cover for OSA interfaces, such as CPAP machine masks, that militates against the formation of condensation within a mask of a wearer and does not contact the wearer.

### SUMMARY OF THE INVENTION

Concordant and congruous with the present invention, a cover for OSA interfaces, such as CPAP machine masks, that militates against the formation of condensation within a mask of a wearer and does not contact the wearer has surprisingly been discovered.

In an embodiment of the invention, a cover for a CPAP mask comprises a main body formed from an insulating material, the main body having a first aperture adapted to receive an elbow of a CPAP mask and a pair of apertures adapted to receive one of a headgear clip and a strap of the CPAP mask; and a means for attaching disposed on the main body configured to attach a portion of the main body to a portion of the CPAP mask.

In another embodiment of the invention, a cover for a CPAP mask comprises a main body having a perimeter, the main body formed from an insulating material, the main body having a first aperture adapted to receive an elbow of a CPAP mask and a pair of indicia indicating where a pair of corresponding apertures should be opened, the apertures adapted to receive one of a headgear clip and a strap of the CPAP mask; and a means for attaching disposed on the main body configured to attach a portion of the main body to a portion of the CPAP mask.

In another embodiment of the invention, a cover for a CPAP mask comprises a main body having a perimeter cooperating with an elastic material adapted to hold the cover on a frame of the CPAP mask, the main body formed from an insulating material, the main body having a first aperture adapted to receive an elbow of a CPAP mask and a pair of apertures adapted to receive one of a headgear clip and a strap of the CPAP mask; and a means for attaching disposed on the main body configured to attach a portion of the main body to a portion of the CPAP mask.

### BRIEF DESCRIPTION OF TEE DRAWINGS

The above, as well as other advantages of the present invention, will become readily apparent to those skilled in the art from the following detailed description of a preferred embodiment when considered in the light of the accompanying drawings in which:

FIG. 1 shows an example of a nasal CPAP mask as known in the art worn by a wearer;

FIG. 2 is an exploded view of the nasal CPAP mask of FIG. 1;

FIG. 3 is a front view of a CPAP mask cover according to an embodiment of the invention;

FIG. 4 is a back view of the CPAP mask cover of FIG. 3;

FIG. 5 is a perspective view the CPAP mask cover of FIGS. 3 and 4 disposed on a CPAP mask according to an embodiment of the invention;

FIG. 6 is a front view of a CPAP mask cover according to another embodiment of the invention;

FIG. 7 is a front view of a CPAP mask cover according to another embodiment of the invention; and

FIG. 8 is a front view of a Rill-face CPAP mask cover according to another embodiment of the invention.

FIG. 9 is a top view of end portions of a sheath for a hose in communication with a CPAP machine according to another embodiment of the invention.

### DETAILED DESCRIPTION OF EXEMPLARY EMBODIMENTS OF THE INVENTION

The following detailed description and appended drawings describe and illustrate various exemplary embodiments of the invention. The description and drawings serve to enable one skilled in the art to make and use the invention, and are not intended to limit the scope of the invention in any manner.

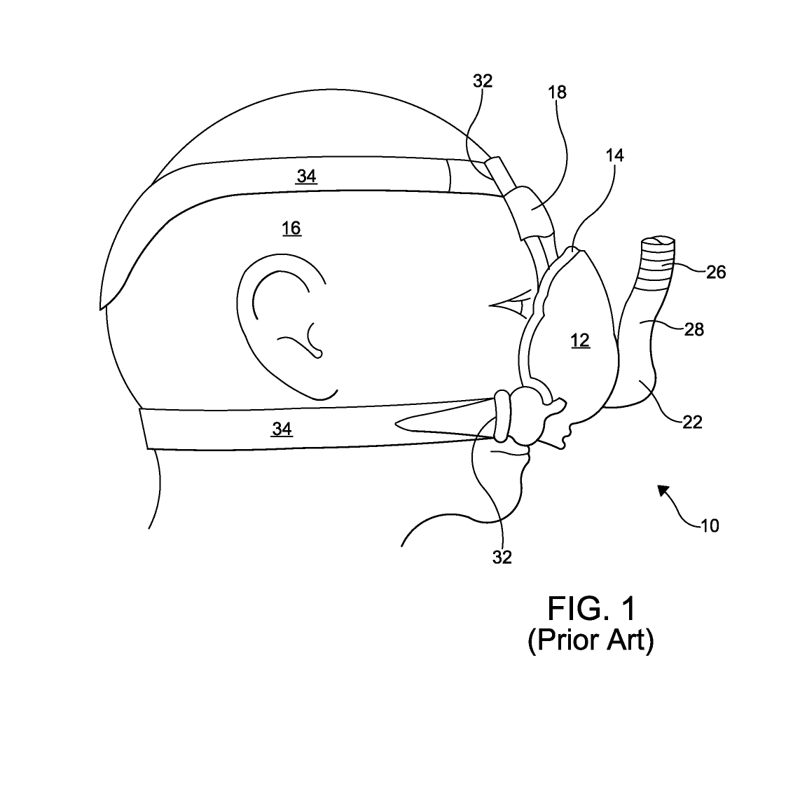

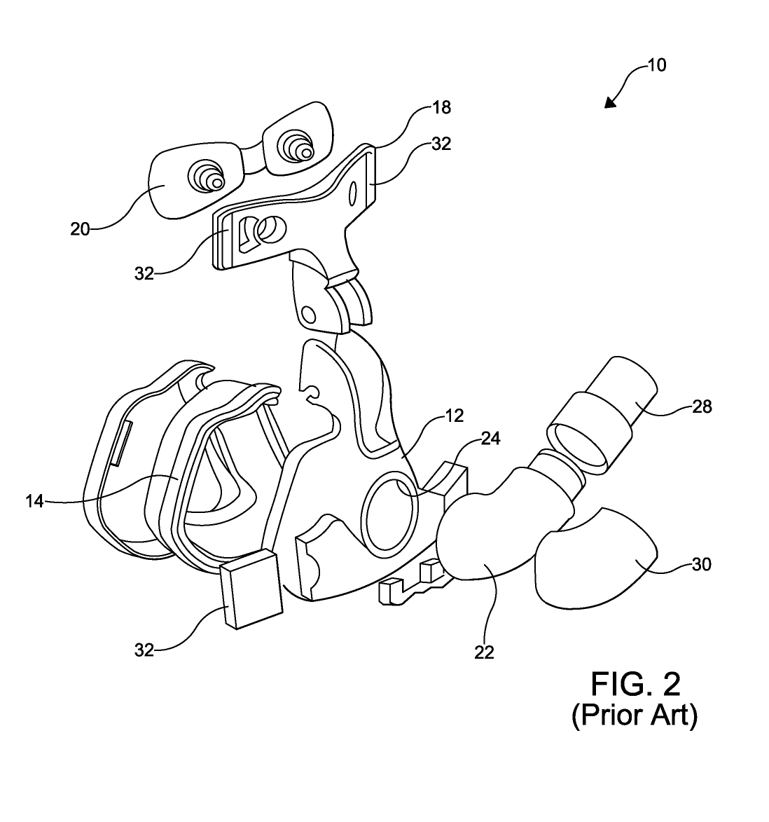

FIGS. 1 and 2 show a typical nasal CPAP mask 10 as known in the art. The CPAP mask 10 includes a mask frame 12 having a cushion 14 that contacts a face of a wearer 16. The cushion 14 forms a seal between the face and the mask frame 12. The mask frame 12 includes a forehead support 18 with a forehead support pad 20 adapted to provide comfort when the forehead support 18 contacts the forehead of the wearer 16. The forehead support 18 is adapted to properly align the CPAP mask 10 on the face of the wearer 16. The CPAP mask 10 further includes a removable elbow 22 that snaps or clicks into (or over) an aperture 24 formed in the mask frame 12. The elbow 22 provides fluid communication between the CPAP machine (not shown) via a hose 26. The elbow 22 may include a swivel 28 adapted to facilitate the rotation of the hose 26 when the wearer 16 moves. The elbow 22 includes a plurality of perforations or apertures (not shown) to provide venting. A selectively removable vent cover 30 having a filter therein may be attached to the elbow 22 to cover the apertures to minimize noise caused by a flow of air therethrough. The mask frame 12 and the forehead support 18 include headgear clips 32 adapted to cooperate with straps 34 that wrap around the head of the wearer 16 to hold the CPAP mask 10 in place. The forehead support 18 may alternatively include slots adapted to cooperate with straps 34 that wrap around the head of the wearer 16 to hold the CPAP mask 10 in place.

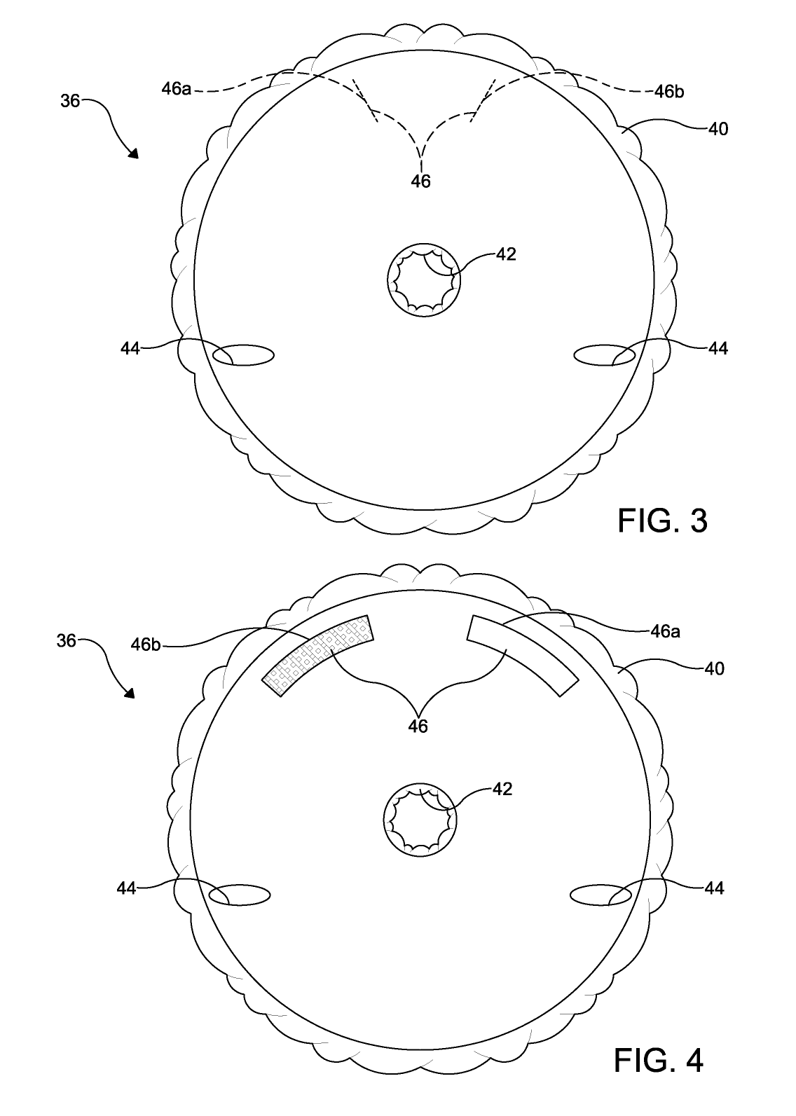

FIGS. 3 and 4 show a CPAP mask cover 36 according to an embodiment of the invention. The cover 36 includes a main body 38 having a generally substantially circular shape. The main body 38 may have a substantially triangular shape or any other shape adapted to cover at least the mask frame 12 of a CPAP mask 10, as desired. The cover 36 is formed from a pliable or flexible material having insulating properties, such as a cotton, a cotton/poly blend, a cotton fleece, a wicking material, a flexible plastic or a rubber, for example, as desired. The cover 36 may also be formed from a less-expensive and disposable material, such as gauze, tissue, paper, or a paper blend material, for example, as desired.

A perimeter 40 of the cover 36 is formed from an elastic material or includes an elastic material sewn therein. A first aperture 42 formed in the main body 38 of the cover 36 and is formed in a center thereof. The aperture 42 has a substantially circular shape adapted to receive the elbow 22 of the CPAP mask 10. The aperture 42 may have any shape, as desired. As shown, to form a fluid tight seal between the main body 38 and the elbow 22, a perimeter of the aperture 42 may also be formed from an elastic material or may include an elastic material sewn therein. The aperture 42 may not include an elastic material, as desired. The aperture 42 may be formed in any portion of the main body 38, as desired.

A pair of apertures 44 formed in the main body 38 are adapted to receive headgear clips 32 of the CPAP mask 12 or the straps 34. The apertures 44 may be horizontally oriented as shown in FIG. 3 or vertically oriented (not shown), as desired. The apertures 44 may be formed in the main body 38 inward of the perimeter 40, as shown in FIG. 3, or the apertures 44 may extend to the perimeter 40 with a portion thereof closeable with a hook-and-loop tape (not shown) or the like to form the apertures. The apertures 44 may be open, or the apertures may be unopened but defined by an indicia, such as a seam, for example, to facilitate the proper location of the apertures 44 by an opening of the apertures 44 by the user to ensure the proper dimensions of the apertures 44 for disposal on the particular CPAP mask of the user.

The cover 36 also includes a means for attaching 46 disposed on a bottom thereof best shown in FIG. 4. It is understood that the means for attaching 46 may be disposed on a top thereof (similar to those of FIGS. 6-8), as desired. The means for attaching 46 is configured to wrap around a portion of the forehead support 18 between the support pads 20 and the mask frame 12. The portion is typically disposed above the bridge of the nose of the user 16. The means for fastening 46 includes a first portion 46a that cooperates with a second portion 46b to hold the top of the cover 36 in place around the portion of the forehead support 18. When the cover 36 is on the CPAP mask 10, the portion of the main body 38 adjacent the portion of the forehead support 18 contacts the forehead support 18 and wraps therearound to hold the top of the cover 36 in place. The portions 46a, 46b of the means for fastening 46 may include snaps, hook-and-loop tape, a button and corresponding hole, reusable adhesive material, and the like, for example.

The top of the mask cover 36, as shown in FIGS. 3-5, includes the elastic perimeter 40 cooperating with the means for fastening 46 to hold the cover 36 in place. However, the top of the mask cover 36 may not include elastic or the back of the cover 36 may not include a means for fastening 46 leaving only one or the other for holding the cover 36 in place, as desired.

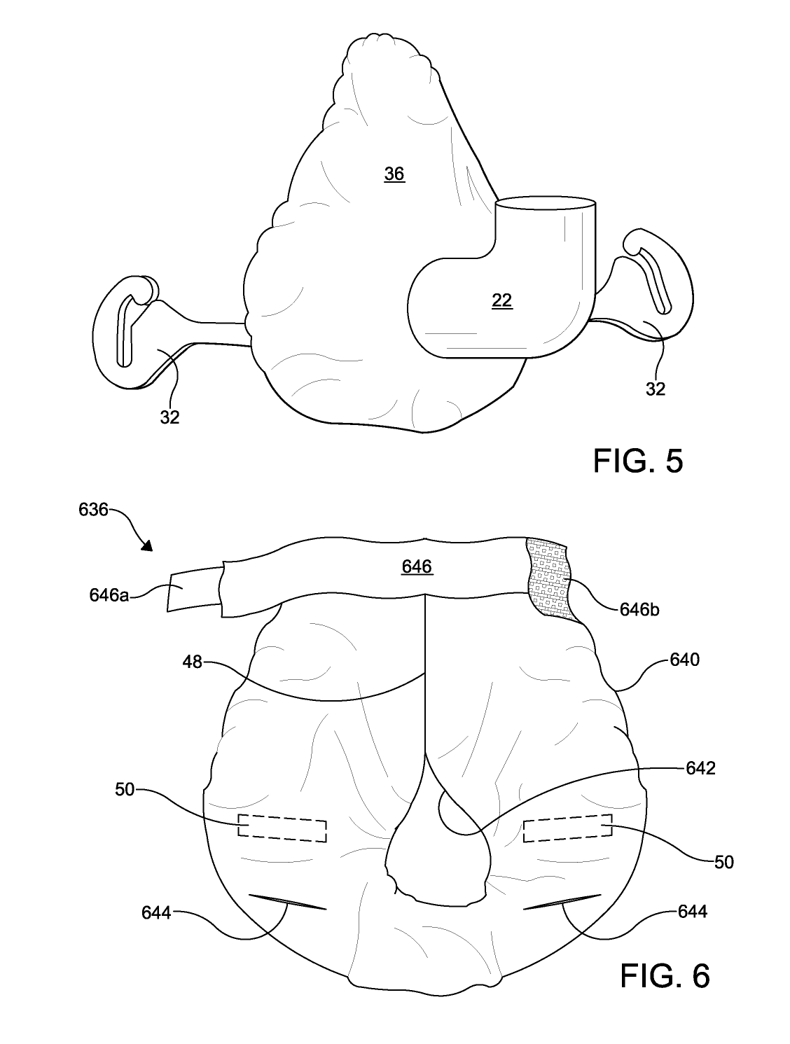

To place the cover 36 over the CPAP mask 10 as shown in FIG. 5, the elbow 22 is disposed through the aperture 42 and the headgear clip(s) 32 of the mask frame 12 are disposed through the apertures 44, if the straps 34 are not attached to the CPAP mask 10. If the straps 34 are attached to the headgear clip(s) and/or slots 32, one end of each strap 34 may be removed from the CPAP mask 10, the end of each strap 34 disposed through the apertures 44, and the end of each strap 34 then reattached to the CPAP mask 10. The elastic perimeter 40 of the cover 36 is disposed around the mask frame 12 without the cover 36 contacting the face of the wearer 16. The elastic perimeter 40 may contact the mask cushion 14, for example, to avoid contacting the face of the wearer 16. The means for attaching 46 is disposed adjacent the portion of the forehead support 18, and the portion of the cover 36 on which the means for attaching 46 is disposed is wrapped around the portion of the forehead support 18 until the first portion 46a of the means for attaching 46a engages the second portion 46b of the means for attaching 46b to hold the top of the cover 36 in place. The means for attaching 46 and the elastic perimeter 40 cooperate to hold the cover 36 in place over the mask frame 12 without the cover 36 contacting the wearer 16 and without affecting the seal formed between the cushion 14 and the face of the wearer 16. The steps required to place a cover 36 over a full CPAP mask (not shown) are similar to the steps described hereinabove except that the cover 36 is larger and covers a mask that covers both the nose and the mouth of the wearer 16.

During use of the CPAP machine, the wearer 16 places the CPAP mask 10 with the cover 36 disposed thereon over the nose (for a nasal mask) or over the nose and mouth (for a full CPAP mask) and turns on the CPAP machine. Because the cover 36 is formed from an insulating material, the CPAP mask 10 and its mask frame 12 are maintained at or near the same temperature as the breath of the user, and the cooler ambient temperature does not cool the CPAP mask 10 and/or the mask frame 12 causing the breath of the wearer 16 to condense on an interior of the CPAP mask 10. If the cover 36 is formed from a wicking material, any condensation that may form on an exterior of the CPAP mask 10 may be wicked away from the CPAP mask 10. Favorable results have been obtained by a wearer 16 using the CPAP mask 10 and cover 36 in a room having an ambient temperature of 68° F.

FIG. 6 shows a CPAP mask cover 636 according to another embodiment of the invention. The embodiment of FIG. 6 is similar to the mask cover 36 of FIGS. 3-5 except as described hereinafter. Similar to the structure of FIGS. 2 and 3, FIG. 6 includes the same reference numerals with a leading 6 to denote similar structure.

Unlike the mask cover 36, the mask cover 636 is substantially ovular in shape rather than substantially round, and a means for attaching 646 is disposed on a top thereof rather than on a back thereof. The means for attach 646 similarly includes a first portion 646a that cooperates with a second portion 646b to hole the top of the cover 636 in place on a CPAP mask. The portions 646a, 646b of the means for fastening 646 may alternatively include snaps, a button and corresponding hole, reusable adhesive material, and the like, for example. The mask cover 636 includes a seam 48 that may be sewn closed or that may be selectively closed using hook and loop tape or the like. The cover 636 further includes a second pair of apertures 50. The second apertures 50 may be horizontal or vertical and located anywhere on the cover 636 desired. As shown in FIG. 6, the second apertures 50 are closed but the location thereof is defined by indicia, such as stiches (or seams) (as shown) or markings, lettering, or another graphic. As described hereinabove, the apertures 50 may be opened by the user to ensure a snug fit. It is understood that the apertures 644 may be similarly closed with an indicia regarding the location of the apertures 644, as desired. The second apertures 50 facilitate use of the cover 636 with a wider range of CPAP masks.

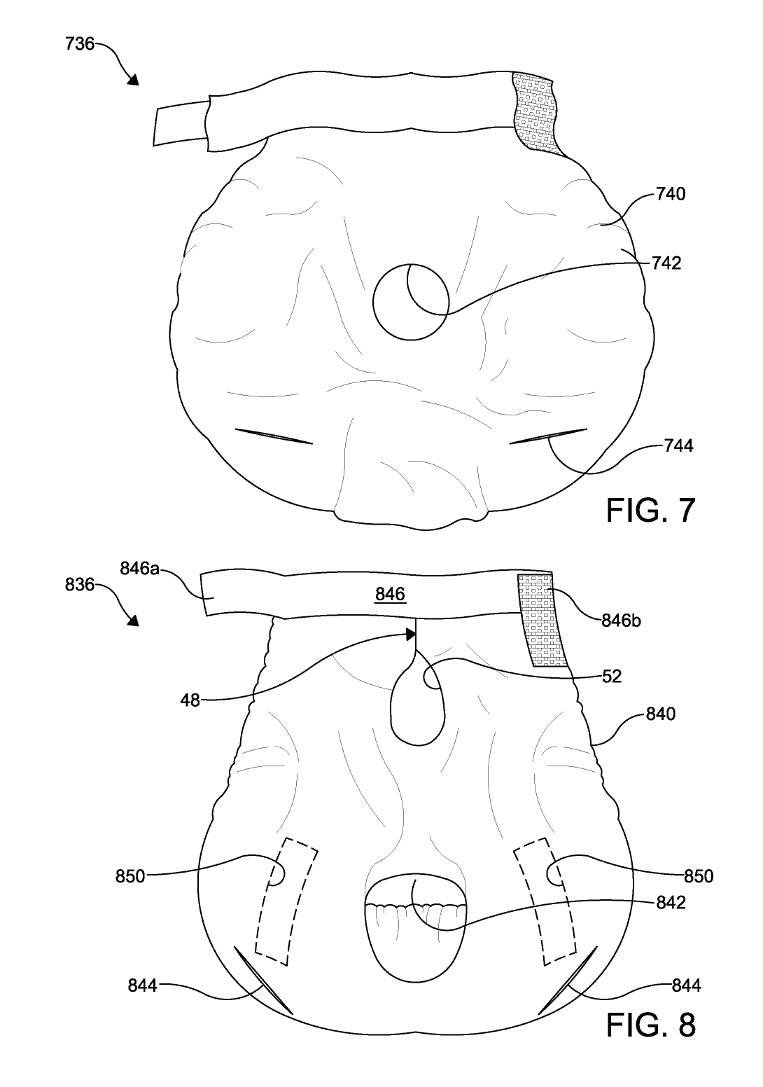

FIG. 7 shows a CPAP mask cover 736 according to another embodiment of the invention. The embodiment of FIG. 7 is similar to the mask cover 636 of FIG. 6 except as described hereinafter. Similar to the structure of FIG. 6, FIG. 7 includes the same reference numerals with a leading 7 to denote similar structure. Unlike the mask cover 636 of FIG. 6, the mask cover 736 does not include the seam 48. Accordingly, the mask cover 736 has a substantially round body rather than an ovular body.

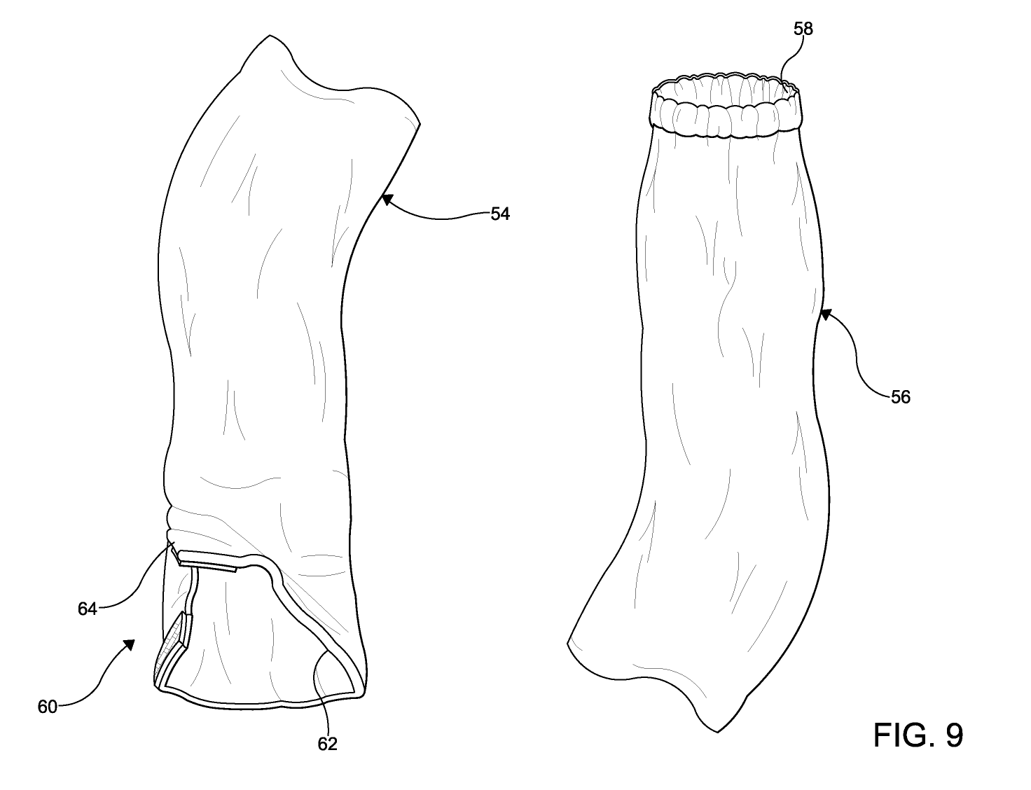

FIG. 8 shows a full-face CPAP mask cover 836 according to another embodiment of the invention. The embodiment of FIG. 8 is similar to the mask cover 636 of FIG. 6 except as described hereinafter. Similar to the structure of FIG. 6, FIG. 8 includes the same reference numerals with a leading 8 to denote similar structure.

As shown in FIG. 8, the apertures 44 are diagonally disposed rather than horizontal (as shown in FIG. 6), and the second apertures 850 of the mask cover 836 are vertically oriented rather than horizontal (as shown in FIG. 6). Furthermore, the mask cover 836 includes a second aperture 52 disposed adjacent a means for attachment 846. By including two pairs of apertures 44, 50 (or two pairs of indicia indicating where apertures should be formed by a user) and two apertures 842, 52, the mask cover 838 is a universal mask that may be used with full-face CPAP masks, nasal CPAP masks, and different variations of such masks that a user may have. To form a fluid tight seal between the main body 838 and the elbow 22, a perimeter of the apertures 842, 52 may also be formed from an elastic material or may include an elastic material sewn therein. As shown, the apertures 842, 52 do not include an elastic material.

FIG. 9 shows a sheath 54 for covering the hose 26 of a CPAP machine. The sheath 54 is formed from a pliable or flexible material having insulating properties, such as a cotton, a cotton/poly blend, a cotton fleece, a wicking material, a flexible plastic or a rubber, for example, as desired. The sheath 54 may also be formed from a less-expensive and disposable material, such as gauze, tissue, paper, or a paper blend material, for example, as desired. The sheath is adapted to maintain a temperature of the air flowing through the hose 26 to militate against condensation on an interior of the hose and/or to maintain the temperature of the air through the hose 26 to ensure liquid does not condense on an interior of the 12 mask.

The sheath 54 includes a first end 56 having an aperture 58. The aperture 58 is defined by a portion of the sheath 54 formed from or including an elastic material. A second end 60 of the sheath 54 includes an aperture 62. The second end 60 further includes a means for fastening 64 adapted to selectively adjust a size of the aperture 62. As shown in FIG. 9, the means for fastening 64 includes hook-and-loop tape, but the means for fastening 64 may include snaps, a button and corresponding hole, reusable adhesive material, and the like, for example. While the sheath 54 shown in FIG. 9 is formed separate from the mask covers 36, 636, 736, 836 described herein, it is understood that the sheath 54 may be integrally formed with the covers 36, 636, 736, 836, as desired. Alternatively, the sheath 54 may be releasably coupled to the covers 36, 636, 736, 836 by a means for fastening (not shown), such as hook-and-loop tape, snaps, a button and corresponding hole, reusable adhesive material, and the like, for example.

In use, the hose 26 is disposed inside of the sheath 54 through the apertures 58, 62 with the first end 56 adjacent the aperture 42, 642, 742, 842, 52 of the cover 36, 636, 736, 836 disposed on the CPAP mask 10. The elastic of the first end 56 holds the sheath 54 in place and adjacent the mask 10 and militates against movement thereof away from the mask 10. The sheath 54 is expanded and, if necessary, stretched so that the second end 60 is spaced apart from the first end 56, The means for fastening 64 is then utilized to minimize a diameter of the aperture 62 to ensure the second end 60 remains spaced apart from the first end 56. It is understood that the first end 54 and the second end 60 may be reversed in orientation with the second end 60 adjacent the mask 10.

From the foregoing description, one ordinarily skilled in the art can easily ascertain the essential characteristics of this invention and, without departing from the spirit and scope thereof, can make various changes and modifications to the invention to adapt it to various usages and conditions.

I claim:

1. A cover for a CPAP mask comprising:
a main body formed from an insulating material, the main body having a first aperture adapted to receive an elbow of a CPAP mask and a pair of apertures adapted to receive one of a headgear clip and a strap of the CPAP mask; and
a means for attaching disposed on the main body configured to attach a portion of the main body to a portion of the CPAP mask.

2. The cover of claim 1, wherein the main body has a perimeter cooperating with an elastic material adapted to hold the cover on a frame of the CPAP mask.

3. The cover of claim 2, wherein the elastic material is sewn into the perimeter.

4. The cover of claim 2, wherein the perimeter is formed from the elastic material.

5. The cover of claim 1, wherein the insulating material is one of a cotton, a cotton/poly blend, and a disposable material.

6. The cover of claim 1, wherein the means for attaching is disposed on a back of the main body.

7. The cover of claim 1, wherein the means for attaching is disposed on a top of the main body.

8. The cover of claim 1, further comprising a sheath for receiving a hose in fluid communication with the CPAP mask.

9. The cover of claim 8, wherein the sheath is formed separate from the cover and having a first end formed from or including an elastic material and a second end including a means for attachment.

10. The cover of claim 8, wherein the sheath is integrally formed with the cover.

11. The cover of claim 8, wherein the pair of apertures are unopened and defined by indicia regarding where a user should open the apertures.

12. The cover of claim 1, wherein the main body includes a second pair of apertures adapted to receive one of a headgear clip and a strap of the CPAP mask.

13. The cover of claim 1, wherein the main body has a second aperture adapted to receive an elbow.

14. A cover for a CPAP mask comprising:
a main body having a perimeter, the main body formed from an insulating material, the main body having a first aperture adapted to receive an elbow of a CPAP mask and a pair of indicia indicating where a pair of corresponding apertures should be opened, the apertures adapted to receive one of a headgear clip and a strap of the CPAP mask; and
a means for attaching disposed on the main body configured to attach a portion of the main body to a portion of the CPAP mask.

15. The cover of claim 14, wherein the main body includes a second pair of apertures adapted to receive one of a headgear clip and a strap of the CPAP mask.

16. The cover of claim 15, wherein the main body has a second aperture adapted to receive an elbow.

17. The cover of claim 14, wherein the elastic material is sewn into the perimeter.

18. The cover of claim 14, wherein the perimeter is formed from the elastic material.

19. A cover for a CPAP mask comprising:
a main body having a perimeter cooperating with an elastic material adapted to hold the cover on a frame of the CPAP mask, the main body formed from an insulating material, the main body having a first aperture adapted to receive an elbow of a CPAP mask and a pair of apertures adapted to receive one of a headgear clip and a strap of the CPAP mask; and
a means for attaching disposed on the main body configured to attach a portion of the main body to a portion of the CPAP mask.

20. The cover of claim 19, further comprising a sheath for receiving a hose in fluid communication with the CPAP mask, the sheath formed separate from the cover and having a first end formed from or including an elastic material and a second end including a means for attachment.
